(19) United States
(12) Patent Application Publication
(10) Pub. No.: US 2026/0123822 A1
(43) Pub. Date: May 7, 2026

(54) STERILE SHEATH FOR PERFORMING NOTES PROCEDURE

(71) Applicant: The Feinstein Institutes for Medical Research, Manhasset, NY (US)

(72) Inventor: Larry S. Miller, Manhasset, NY (US)

(21) Appl. No.: 19/376,142

(22) Filed: Oct. 31, 2025

Related U.S. Application Data
(60) Provisional application No. 63/715,007, filed on Nov. 1, 2024.

(51) Int. Cl. A61B 1/00; A61B 1/018; A61B 1/273

(52) U.S. Cl. CPC A61B 1/00142; A61B 1/00082; A61B 1/018; A61B 1/2736

(57) ABSTRACT

A device creates a sterile passage to perform natural orifice transluminal endoscopic surgery (NOTES). A balloon tube is connected with a fluid-impervious shroud to define an interior passage that is continuous and that can exclude pathogens. Two torus-shape balloons are arranged axially along the outside surface of the balloon tube and define a circumferential gap. In an exemplary use, an endoscope/laparoscope is inserted through the shroud and through the balloon tube. The endoscope/laparoscope, surrounded by the shroud, is inserted through a patient's mouth and advanced to a position in the stomach adjacent where a procedure will be performed in the peritoneum. The ballon tube is positioned through an incision in the stomach wall so that the wall is positioned in the gap between the balloons. The balloons are inflated to seal the balloon tube with the stomach wall to create a sterile pathway for surgical instruments.

This application claims priority under 35 U.S.C. §119 to U.S. Provisional Patent Application No. 63/715,007 , filed on Nov. 1, 2024. The disclosure of that application is incorporated herein by reference.

### BACKGROUND

### Field

This disclosure relates to a sheath for a surgical instrument used to facilitate performance of natural orifice transluminal endoscopic surgery (NOTES). In particular, the disclosure relates to a sheath that provides a sterile path to deploy instruments via a patient's natural bodily opening and through an anatomical wall into a body cavity. According to one embodiment, such a sheath is used to facilitate access to a patient's peritoneal cavity via the patient's mouth while preventing pathogens and contaminants that may be present in the patient's gastrointestinal tract from flowing into the peritoneum. According to other embodiments, a sheath is provided to facilitate NOTES procedures via other natural orifices such as the rectum, vagina, anus or nose. According to other embodiments two or more sheaths could be used at the same or different anatomical location to perform procedures that are similar to standard laparoscopic procures using multiple ports and laparoscopes.

NOTES procedures allow surgeons to access body cavities and perform minimally invasive surgical (“MIS”) procedures without the need for an incision through the patient's skin. Such procedures may reduce surgical pain and trauma to the patient, reduce the chance of infection through an external surgical wound, and reduce complexity of certain procedures. Such procedures may access body cavities via a natural opening such as the mouth, the vagina, the anus, the rectum, or other opening.

In NOTES procedures, an endoscope or laparoscope is inserted through the natural orifice, for example, the patient's mouth. The endoscope/laparoscope is advanced to the part of the stomach, duodenum, or intestine adjacent to the part of the abdominal cavity selected for the surgical procedure. According to known techniques, an incision is made into the peritoneal cavity through the stomach, esophageal, colon, small intestine, vagina or rectal wall and endoscopic or laparoscopic instruments are manipulated through the incision to perform the procedure in the abdominal cavity.

Known NOTES procedures may allow pathogens or other contaminants to move through the incision from an unsterilized part of the patient's body, e.g., the stomach, esophagus, vagina, or rectum, into an internal body cavity, e.g., the peritoneal cavity, leading to potential infection. This risk of infection may limit the use of NOTES techniques. Thus, there is a need for a device and a method that can provide a sterile pathway to access a body cavity via a natural orifice.

### SUMMARY

According to one aspect of the disclosure, a sheath is provided that can be deployed to create a sterile pathway for endoscopic or laparoscopic instruments to access internal body cavities, such as the peritoneal cavity via a natural orifice such as the mouth. According to one embodiment, the sheath includes a shroud that will form a sterile passageway for instruments performing a NOTES procedure. A balloon tube is connected with a distal portion of the shroud and a seal is formed between the shroud and the outside surface of the balloon tube. The balloon tube defines a central passage sized to permit endoscopic or laparoscopic instruments to pass through. On the outer surface of the balloon tube are two balloons separated by a circumferential gap and extending around the circumference of the tube. According to one embodiment, the shroud is connected with, and sealed to, the circumference of the balloon tube at a location proximal of the proximal-most balloon. A distal end portion of the sheath extends distally from the point where it is connected with the balloon tube and encloses the distal end of the balloon tube.

According to one embodiment, the shroud extends proximally a sufficient length that, when deployed, the shroud reaches from the selected site of the NOTES procedure, through the GI tract and out of the patient's body through a natural orifice, e.g., the patient's mouth.

According to one embodiment, the balloon tube includes a gasket to reduce the flow of gases through the tube to facilitate insufflation once the device has been deployed, as will be discussed below. According to one embodiment, the internal surfaces of the sheath including the shroud and balloon tube are sterilized and stored to maintain sterility, using techniques known to those of skill in the field of the disclosure. According to another embodiment, the sheath and balloon tube may not be maintained in a sterile condition and may be sterilized just prior to or during a surgical procedure

Such a device may be used to perform a NOTES procedure, for example, in the stomach to perform a procedure in the peritoneum. According to one embodiment a surgical procedure is performed on tissues in the peritoneum through the wall of the stomach. Hetastarch or other suitable substance is injected into the stomach wall to raise a bleb separating the mucosa and submucosa from the muscle layer. An incision is made in the bleb and an unsterilized endoscope or laparoscope is inserted through the incision to create a tunnel between the sub mucosa and the muscle layer. According to one embodiment, the tunnel is about 5 centimeters (cm) to 10 cm long. The unsterilized endoscope/laparoscope is withdrawn.

A sterilized endoscope/laparoscope is inserted through the shroud so that its distal end is engaged with the balloon tube. According to one embodiment, the sterile endoscope/laparoscope connects with features of the balloon tube so that the balloon tube can be manipulated by the practitioner operating the endoscope/laparoscope. According to one embodiment, the distal end of the endoscope/laparoscope extends from the distal end of the balloon tube.

According to an alternative embodiment, the endoscope/laparoscope and the sheath are non-sterile prior to the surgical procedure and are sterilized after the endoscope/laparoscope is inserted into the sheath. According to one embodiment, the shroud is made from a material that allows the passage of sterilizing radiation, for example ultraviolet light at a suitable wavelength, e.g., UVc radiation. An endoscope/laparoscope equipped with a source of sterilizing radiation is inserted through the shroud. Illumination from the light source reduces or eliminates pathogens on the inside surface of the shroud and the balloon tube. According to another embodiment, a disinfecting liquid is used to sterilize or partially sterilize the mucosa and the tunnel. This liquid can be, but is not limited, to hydrogen peroxide or betadine.

Once the endoscope/laparoscope is engaged with the balloon tube, the endoscope/laparoscope surrounded by the shroud is inserted through the patient's mouth, with the open, proximal end of the shroud located away from the mouth to maintain sterility of the endoscope/laparoscope and the interior of the shroud.

According to one embodiment, the endoscope/laparoscope includes a light source that delivers sterilizing radiation, e.g., UVc light, sufficient to sterilize or partially sterilize portions of the stomach lining. The light source delivers sterilizing radiation to the bleb and portions of the stomach lining surrounding the incision to reduce or eliminate pathogens in and around the incision. Apparatus and methods for delivering sterilizing radiation can be found in U.S. Pat. No. 11,554,187, issued on Jan. 17, 2023, which is incorporated herein by reference in its entirety. According to another embodiment, a disinfecting liquid is used to sterilize the mucosa and the tunnel. This liquid can be, but is not limited, to hydrogen peroxide or betadine.

According to one embodiment, once the area of the incision is sterilized, the distal portion of the shroud that surrounds the distal end of the balloon tube is cut or torn so that the distal portion of the endoscope/laparoscope and balloon tube are not surrounded by the distal portion of the shroud. According to one embodiment, a cutting instrument, for example, a needle knife, is advanced through an instrument channel in the endoscope/laparoscope to cut open the distal end of the shroud that surrounds the distal end of the balloon tube. According to another embodiment, the distal portion of the shroud includes perforations that can be broken by advancing the endoscope/laparoscope distally to rupture the perforated section.

Once the distal portion of the shroud is opened, the endoscope/laparoscope is manipulated so that the balloon tube is advanced through the incision and along the tunnel created within the bleb. Instruments, such as a needle knife are advanced through the endoscope/laparoscope to create an incision through the stomach wall. The endoscope/laparoscope is manipulated to advance the balloon tube through the muscle layer so that the distal balloon is in the peritoneum and the proximal balloon is in the tunnel within the stomach. In this arrangement, the stomach wall is positioned in the circumferential gap between the balloons.

The balloons are then inflated. The distal balloon contacts the serosa and the proximal balloon contacts the mucosa to create a seal around the perimeter of the balloon tube. Surgical procedures may now be performed by advancing instruments along the sterile pathway formed by the shroud and balloon tube.

When the procedure is finished the balloons are deflated and the tube is removed. The incision in the muscle layer and mucosa are closed, for example, using surgical clips or endoscopic sutures.

Exemplary embodiments describe methods and apparatus adapted to access a peritoneal cavity via the mouth. The disclosure includes methods and apparatus for accessing other body cavities, such as the pelvic cavity, the thoracic cavity, the cranial cavity, and to access such cavities via another natural orifice, for example, the nose, vagina, or anus.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows an endoscope/laparoscope positioned within a sheath according to an embodiment of the disclosure;

FIG. 2 shows a cross section of a balloon tube and distal portion of a shroud of the sheath of FIG. 1;

FIG. 3 shows an endoscope/laparoscope engaged with the sheath of FIG. 1 with a distal portion of the shroud intact;

FIG. 4 shows an endoscope/laparoscope engaged with the sheath of FIG. 1 with a distal portion of the shroud having been removed or ruptured; and

FIGS. 5-11 show steps for performing a surgical procedure using the sheath of FIG. 1.

### DETAILED DESCRIPTION

As used herein, the term “distal” refers to the direction toward a bodily structure being treated using apparatus according to embodiments of the disclosure. The term “proximal” refers to the direction away from the bodily structure being treated and towards a practitioner manipulating the apparatus.

For ease of description, the user operating the device may also be referred to herein as the “operator,” “healthcare provider,” “medical provider,” or “practitioner.”

While several embodiments are described, the new subject matter described in this patent disclosure is not limited to any one embodiment or combination of embodiments described herein, but instead encompasses numerous alternatives, modifications, and equivalents. In addition, while numerous specific details are set forth in the following description to provide a thorough understanding, some embodiments can be practiced without some or all such details.

Moreover, for the purpose of clarity, certain technical matters that are known in the related art have not been described in detail in order to avoid unnecessarily obscuring the new subject matter described herein. It should be clear that individual features of one or several of the specific embodiments described herein can be used in combination with features of other described embodiments or with other features. Further, like reference numbers and designations in the various drawings indicate like elements.

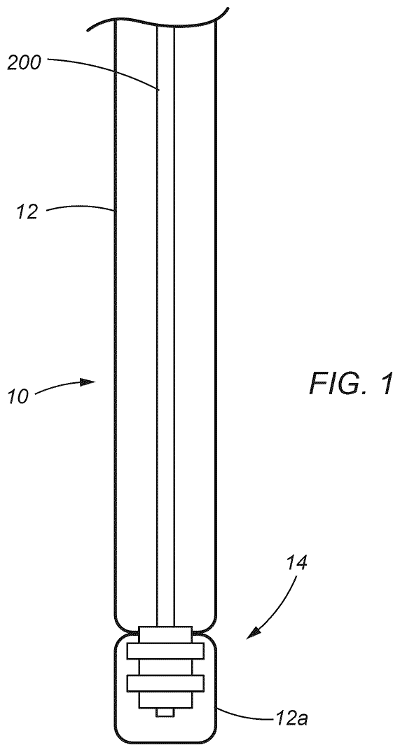

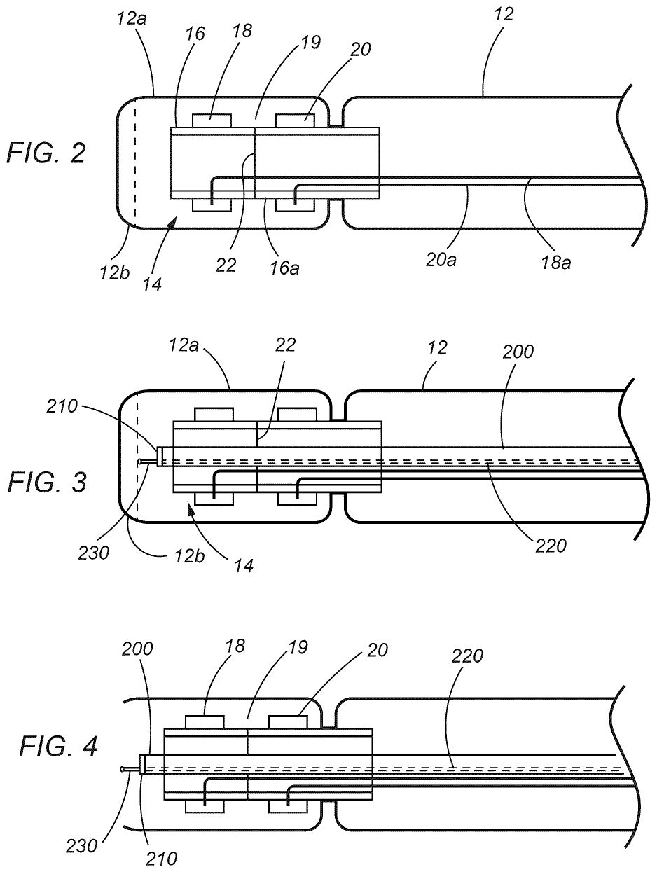

FIG. 1 shows sheath 10 according to an embodiment of disclosure with endoscope/laparoscope 200 extending therethrough. Sheath 10 is formed by balloon tube 14 connected at a distal portion of shroud 12. As shown in FIG. 2, shroud 12 connects with balloon tube 14 to form a seal. A distal portion of shroud 12 forms distal covering 12a that encloses the distal end of balloon tube 14.

As shown in FIG. 2, balloon tube 14 is formed by cylindrical passage 16. Passage 16 has an internal diameter sized to allow laparoscopic or endoscopic instruments to pass through balloon tube 14. Passage 16 has an outside surface 16a. Distal balloon 18 and proximal balloon 20 extend radially around passage 16 along outside surface 16a and define a gap 19 between them. Inflation tubes 18a, 20a extend proximally along the inside of shroud 12. Tubes 18a, 20a are connected with apparatus known in the field of the disclosure to selectively inflate and deflate respective balloons 18, 20.

According to one embodiment, as shown in FIG. 2 shroud 12 is connected with outside surface 16a along a circumference proximal of proximal balloon 20. According to other embodiments, shroud 12 connects with surface 16a in gap 19 between distal balloon 18 and proximal balloon 20. According to another embodiment, shroud 12 connects with surface 16a along a circumference distal of distal balloon 18.

According to one embodiment, gasket 22 extends across the interior of passage 16. During procedures using sheath 10, gasket 22 prevents or reduces the leakage of insufflation gases from within a body cavity undergoing a surgical procedure.

As shown in FIG. 3, according to one embodiment endoscope/laparoscope 200 is advanced through shroud 12 and engages with balloon tube 14. Endoscope/laparoscope 200 extends through gasket 22 and extends out from the distal end of passage 16. According to one embodiment, the fit of endoscope/laparoscope 200 with passage 16 and/or gasket 22 forms a mechanical connection so that a practitioner operating the endoscope/laparoscope can steer balloon tube 14, as will be described below.

Distal covering 12a of shroud 12 may include a perforated or otherwise weakened portion 12b to facilitate opening the distal end of the shroud, as will be described below.

According to one embodiment, endoscope/laparoscope 200 includes light source 210 that emits germicidal radiation to sterilize tissues and other surfaces where a NOTES procedure will take place. According to one embodiment, light source 210 emits light with a wavelength of about 180-280 nanometers (nm). Apparatus and methods for delivering sterilizing radiation can be found in U.S. Pat. No. 11,554,187, issued on Jan. 17, 2023, which is incorporated herein by reference in its entirety. According to one embodiment, light source 210 includes a window that is transmissive to the germicidal radiation.

Instrument passage 220 is provided through endoscope/laparoscope 200. Instruments 230, such as a needle knife may be advanced along instrument passage 220 and operated by the practitioner, as will be described below.

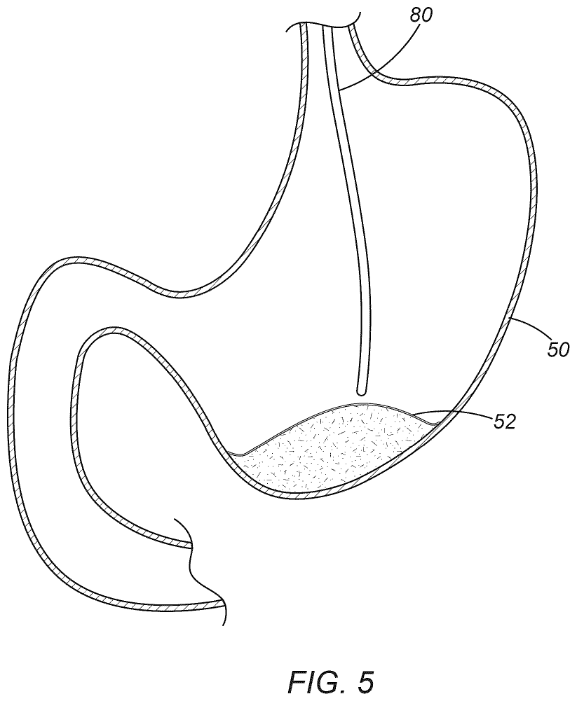

Steps for accessing the peritoneum through the wall of the stomach via the mouth is described according to an embodiment of the disclosure as shown in FIGS. 5-11. The disclosure is not limited to this access route and includes other NOTES procedures known in the field of the disclosure.

As shown in FIG. 5, first endoscope/laparoscope 80 is advanced through the esophagus and into the stomach. First endoscope/laparoscope 80 contacts mucous membranes as it is advanced, potentially collecting pathogens, thus, according to one embodiment, endoscope/laparoscope 80 is non-sterile. Instruments advanced through endoscope/laparoscope 80 are used to form bleb 52 and to inject a solution of hetastarch or other suitable substance between the submucosa and the muscle layer. Instruments advanced through endoscope/laparoscope 80 are used to form an incision in bleb 52.

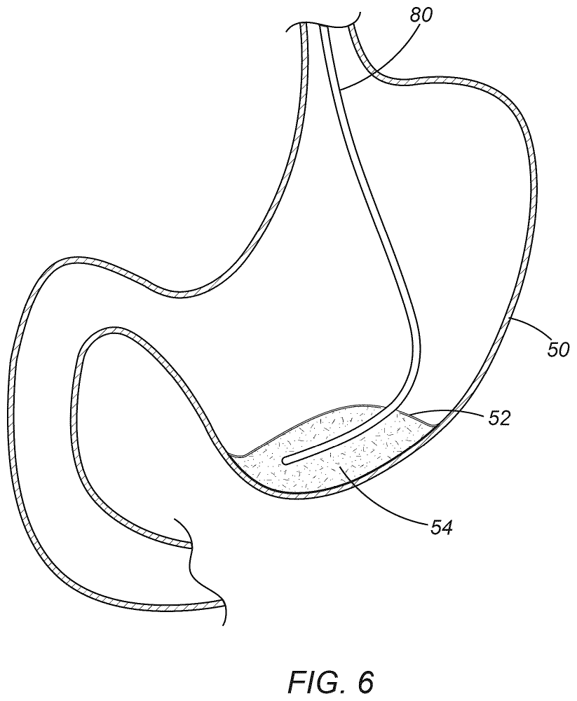

As shown in FIG. 6, endoscope/laparoscope 80 is advanced through the incision and through bleb 52 separating the submucosa and mucosa from the muscle layer to create tunnel 54. According to one embodiment, tunnel 54 may be between about 5 cm and about 10 cm long. Endoscope/laparoscope 80 is withdrawn from the patient.

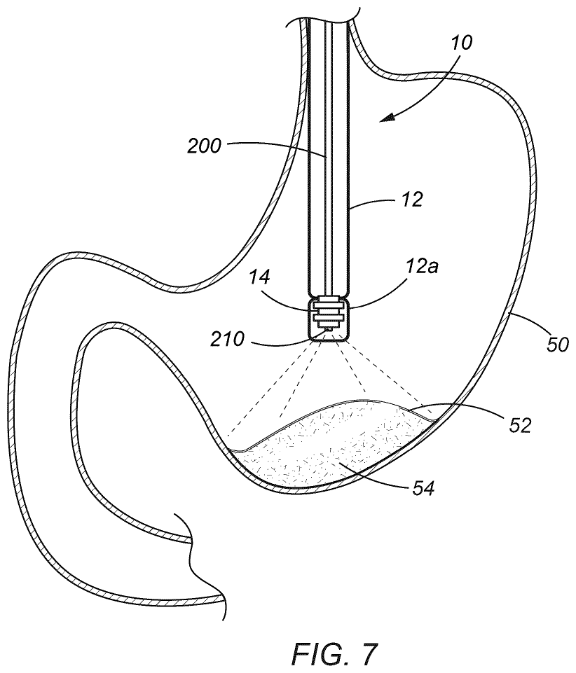

Second endoscope/laparoscope 200 is engaged with sheath 10 as shown in FIG. 3. Endoscope/laparoscope 200 extends through balloon tube 14 so that light source 210 extends from the distal end of endoscope/laparoscope 200 or balloon tube 14. As shown in FIG. 7, endoscope/laparoscope 200 and balloon tube 14 are enclosed in sheath 12 and distal cover 12a and advanced into stomach 50. The distal end of endoscope/laparoscope 200 is positioned near bleb 52. Light source 210 illuminates the incision and the surrounding tissue with germicidal radiation, for example UVc light, reducing or eliminating pathogens on the mucosa surrounding the incision. According to one embodiment, the germicidal radiation also sterilizes distal cover 12a so that pathogens picked up from the patient's mouth and esophogus on the cover are reduced or eliminated. According to another embodiment, a disinfecting liquid is used to sterilize the mucosa and the tunnel. This liquid can be, but is not limited, to hydrogen peroxide or betadine.

According to one embodiment, cutting instrument 230 is advanced through the instrument passage of endoscope/laparoscope 200, as shown in FIG. 3. Cutting instrument 230 is manipulated to cut away distal cover 12a to expose the distal portion of endoscope/laparoscope 200 and balloon tube 14, as shown in FIG. 4. According to another embodiment, distal cover 12a includes a perforated or weakened region 12b, as shown in FIG. 3. Instead of cutting through distal cover 12a, endoscope/laparoscope 200 is advance through balloon tube 14 to press against distal cover 12a to rupture weakened region 12b.

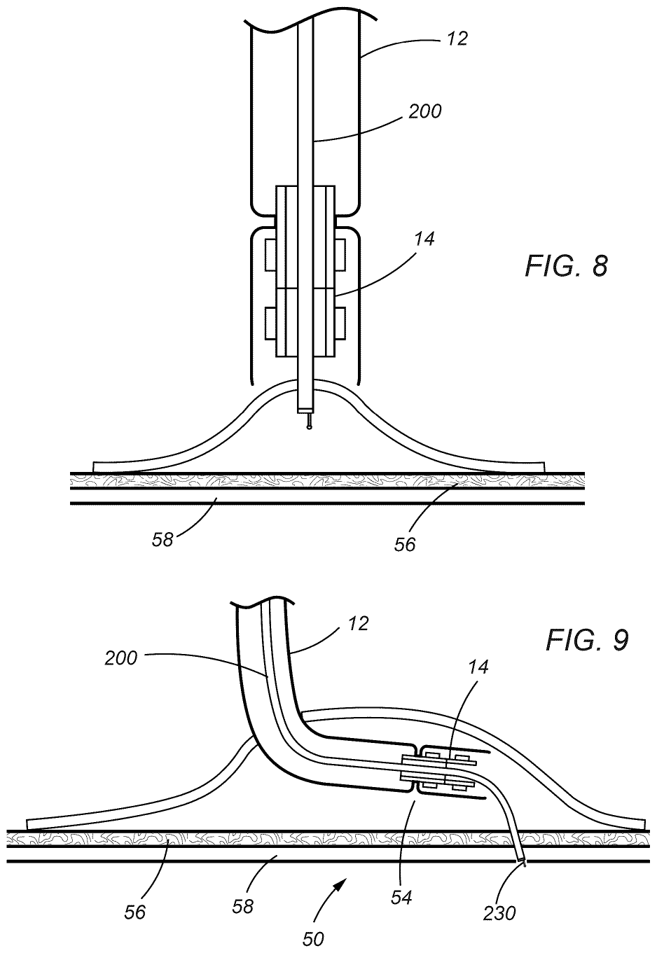

As shown in FIGS. 8 and 9, endoscope/laparoscope 200 and balloon tube 14 are advanced through the incision and along tunnel 54. Shroud 12 extends in the proximal direction through the stomach and out from the natural orifice.

As shown in FIG. 9, endoscope/laparoscope 200 is advanced along tunnel 54 to a position selected by the practitioner for the procedure. Instrument 230 is advanced through instrument passage 220 and is used to create an incision through muscle layer 56 and serosa 58.

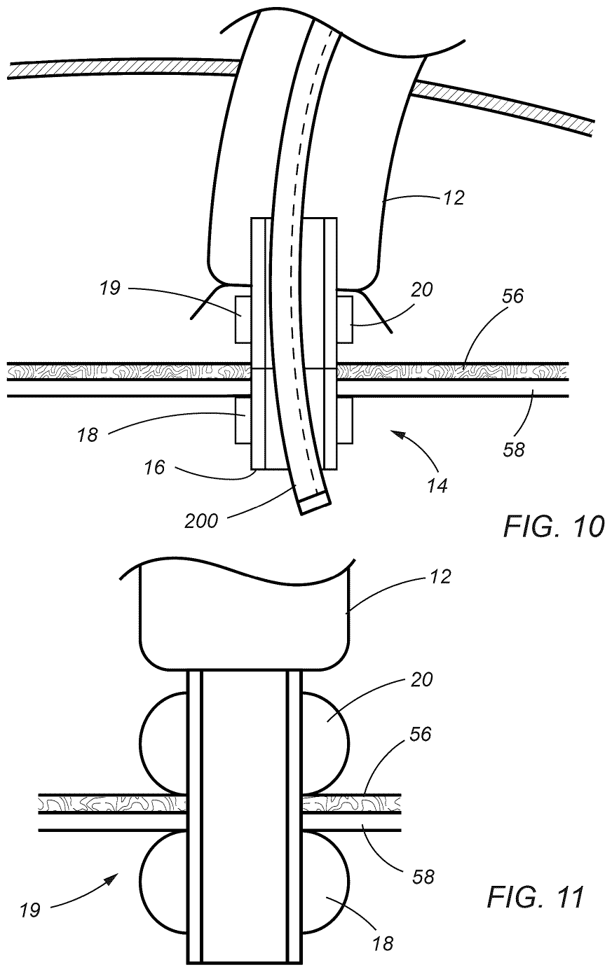

As shown in FIG. 10, endoscope/laparoscope 200 is manipulated to advance balloon tube 14 through the incision through the muscle layer and serosa so that distal balloon 18 is positioned beyond the serosa in the peritoneum and proximal balloon 20 is positioned proximal of muscle layer 56. In this configuration the serosa and muscle layer are positioned in circumferential gap 19.

As shown in FIG. 11, proximal and distal balloons 18, 20 are inflated. According to one embodiment, fluid or gas is delivered via inflation tubes 18a, 20a, as shown in FIG. 2 using a syringe or other method known to those of skill in the field of the disclosure.

When balloons 18, 20 fully expanded, they oppose serosa 58 and muscle layer 56, respectively, and act as a barrier for purposes of sterility. Shroud 12 is sealed with the proximal portion of balloon tube 14 and forms a continuous, sterile pathway to advance laparoscopic instruments into the peritoneum via passage 16 to perform a NOTES procedures.

When the procedure is finished, balloons 18, 20 are deflated and sheath 10 is withdrawn from tunnel 54 and removed from the patient. The incisions are close using sutures, clips or using other techniques known to those of skill in the field of the disclosure.

In the above description, the use of common reference numerals in different examples is for convenience only and is intended to refer to similar components and not necessarily identical components. While illustrative embodiments of the disclosure have been described and illustrated above, it should be understood that these are exemplary of the disclosure and are not to be considered as limiting. Additions, deletions, substitutions, and other modifications can be made without departing from the spirit or scope of the disclosure. Accordingly, the disclosure is not to be considered as limited by the foregoing description.

We claim:

1. A device for facilitating a surgical procedure, comprising:
a fluid-impervious shroud comprising a continuous interior passage;
a balloon tube, wherein the balloon tube comprises an interior passage, an outside surface, a proximal portion and a distal portion; and
a torus-shape proximal balloon and a torus-shaped distal balloon, wherein the proximal and distal balloons are arranged axially along the outside surface,
wherein a distal portion of the shroud is connected with the outside surface along a circumference of the outside surface, and wherein the connection is fluid-impervious.

2. The device according to claim 1, wherein the balloon tube is adapted to engage with a distal end of an endoscope.

3. The device according to claim 1, further comprising a gasket disposed across the interior passage.

4. The device according to claim 1, further comprising a distal cover connected with the balloon tube at the circumference and extending in a distal direction, wherein the distal cover encloses the distal portion of the balloon tube.

5. The device according to claim 4, wherein the distal cover includes a weakened portion adapted to be ruptured by a surgical instrument.

6. The device according to claim 1, wherein the surgical procedure comprises a NOTES procedure.

7. A method for surgically accessing a body cavity through a natural orifice using the device according to claim 1, comprising the steps of:
extending an endoscope through the inner passage of the shroud, wherein a distal end of the endoscopeextends at least partly within the inner passage of the balloon tube;
inserting the endoscope surrounded by the shroud though the natural orifice and along a body channel to a surgical site, wherein pathogens in the body channel are excluded from the inner passage;
positioning the balloon tube through an incision in a tissue wall, wherein the distal balloon is positioned distal of the wall and the proximal balloon is positioned proximal of the wall; and
inflating the proximal and distal balloons, wherein the balloons contact respective proximal and distal sides of the wall and create a fluid-impervious seal between the balloon tube and the wall.

8. The method of claim 7, wherein the endoscope comprises an instrument passage, further comprising
performing a surgical procedure at a location distal of the wall by moving one or more surgical instruments through the instrument passage.

9. The method of claim 7, wherein the endoscope comprises a source of germicidal radiation, wherein the step of extending the endoscope through the inner passage comprises illuminating the shroud with the germicidal radiation, wherein pathogens on within the inner passage are reduced or eliminated.

10. The method of claim 9, wherein the tissue wall is a stomach wall between a stomach and a peritoneum, further comprising the steps of:
forming a bleb below a mucosa of the stomach;
creating an opening through the mucosa into the bleb;
extending an instrument through the opening to form a tunnel;
inserting the balloon tube and the distal portion of the endoscope along the tunnel; and
forming the incision, wherein the incision extends through a muscle layer and a serosa into the peritoneum.
